(19) United States
(12) Patent Application Publication
(10) Pub. No.: US 2019/0204319 A1
(43) Pub. Date: Jul. 4, 2019

(54) Method of Screening Breast Cancer by Using Serum WISP1 Level as a Biomarker

(71) Applicants: Kun-Chun Chiang, Keelung City (TW); Chun-Nan Yeh, Taoyuan City (TW); Horng-Heng Juang, Taoyuan City (TW); Ta-Sen Yeh, Taoyuan City (TW)

(72) Inventors: Kun-Chun Chiang, Keelung City (TW); Chun-Nan Yeh, Taoyuan City (TW); Horng-Heng Juang, Taoyuan City (TW); Ta-Sen Yeh, Taoyuan City (TW)

(73) Assignees: Chang Gung Memorial Hospital, Keelung, Keelung City (TW); Chang Gung University, Taoyuan City (TW)

(21) Appl. No.: 15/857,870

(22) Filed: Dec. 29, 2017

(51) Int. Cl. G01N 33/574

(52) U.S. Cl. CPC G01N 33/57415; G01N 2333/4703; G01N 33/57488

(57) ABSTRACT

A method of screening breast cancer includes collecting serum samples from breast cancer patient; measuring WISP1 level in the serum sample; and comparing the serum WISP1 level of the healthy persons with the WISP1 level in the serum sample of the breast cancer patients. The mean serum WISP1 level is 631.5 pg/ml for healthy persons and the mean serum WISP1 level is 934.5 pg/ml for patients having breast cancer so that WISP1 level of 934.5 pg/ml is configured to be a biomarker in determining whether a subject has high risk of breast cancer occurrence or not.

### BACKGROUND OF THE INVENTION

### 1. Field of the Invention

The invention relates to breast cancer screening and more particularly to a method of screening breast cancer by measuring WNT1-inducible-signaling pathway protein 1 (WISP1) level in a serum sample of a subject so that the measured WISP1 level can be taken as a biomarker for indicating whether the subject has high risk of breast cancer occurrence or recurrence or not.

### 2. Description of Related Art

Breast cancer is cancer that develops from breast tissue. Diagnosis of breast cancer is confirmed by taking a biopsy of the concerning lump. Once the diagnosis is made, further tests are done to determine if the cancer has spread beyond the breast and which treatments it may respond to. In those who have been diagnosed with cancer, a number of treatments may be used, including surgery, radiation therapy, chemotherapy and targeted therapy. However, for those who have been treated in a later stage of breast cancer, the cancer may still spread to other parts of the body.

Early diagnosis of cancer by screening, in time detection of the reoccurrence of cancer for those who have been treated, and evaluation of cancer treatment are three important parts to improve breast cancer treatment and prognosis. However, there is no reliable conventional biomarker for indicating whether a person has breast cancer or not. Conventionally, cancer antigen 15-3 (CA153) and carcino-embryonic antigen (CEA) are used as biomarker for indicating whether a person has breast cancer or not after taking breast cancer screening. However, their sensitivity and specificity with respect to each of early diagnosis of cancer and in time detection of the recurrence of cancer for those who have been treated are poor.

Thus, the need for improvement still exists.

### SUMMARY OF THE INVENTION

It is therefore one object of the invention to provide a method of screening breast cancer comprising the steps of collecting a serum sample from breast cancer patients; measuring WISP1 level in the serum sample; and comparing the serum WISP1 level of healthy persons with the WISP1 level in the serum of breast cancer patients; wherein the mean serum WISP1 level is 631.5 pg/ml for healthy persons and the mean serum WISP1 level is 934.5 pg/ml for breast cancer patients so that serum WISP1 level of 934.5 pg/ml is configured to be a biomarker in determining whether a subject has high risk of breast cancer occurrence or reoccurrence or not by comparing the serum WISP1 level of the subject with the serum WISP1 level of 934.5 pg/ml.

Preferably, the serum WISP1 level of a breast cancer subject after treatment is less than 934.5 pg/ml means that a treatment of surgery, chemotherapy, or targeted therapy for the subject having breast cancer is effective.

Preferably, the serum WISP1 level of a breast cancer subject after treatment is greater than 934.5 pg/ml means that a treatment of surgery, chemotherapy, or targeted therapy for the subject having breast cancer is ineffective, and the subject having high risk of breast cancer recurrence and metastasis.

Preferably, the method is used to evaluate the breast cancer treatment response in the breast cancer patients and recovery therefrom.

Preferably, the measurement of the serum WISP1 level is done by using Enzyme-linked immunosorbent assay (ELISA).

The invention has the following advantages and benefits in comparison with the conventional art:

WISP1 level can quickly evaluate effectiveness of medicine for breast cancer treatment. It is highly possible that a subject may have breast cancer if WISP1 level in his or her serum is equal to or greater than 934.5 pg/ml. Otherwise, it is determined that the subject does not have breast cancer. Sensitivity for diagnosing breast cancer is 57% and specificity for diagnosing breast cancer is 96% as implemented by measuring WISP1 level. It can determine that a subject may have breast cancer if WISP1 level in his or her serum is equal to or greater than a predetermined value (e.g., 934.5 pg/ml). Thus, the WISP1 level can be used a biomarker in determining whether a subject has high risk of breast cancer occurrence or not.

The above and other objects, features and advantages of the invention will become apparent from the following detailed description taken with the accompanying drawings.

### BRIEF DESCRIPTION OF THE DRAWINGS

FIG. 1 is a table showing WISP1 level median of 14 cancerous subjects, WISP1 level median of 31 healthy subjects, and the P value according to the invention;

FIG. 2 is a table showing preoperative and postoperative mean level of WISP1, CA153, and CEA in the serum of 14 cancerous subjects according to the invention;

FIG. 3 is a table showing mean serum WISP1 level of 31 healthy subjects being 675.91 pg/ml, and mean serum WISP1 level average of 14 postoperative cancerous subjects being 631.46 pg/ml according to the invention; and

FIG. 4 shows ROC curve for serum WISP1 levels and the ROC curve is constructed by comparing the healthy persons with the breast cancer patients according to the invention.

### DETAILED DESCRIPTION OF THE INVENTION

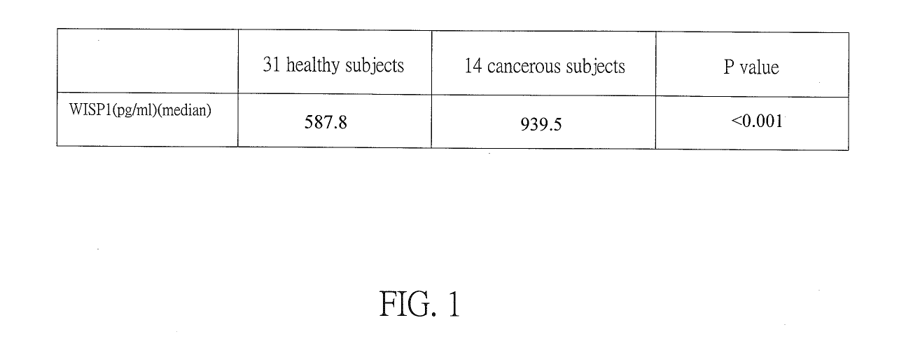

Referring to FIGS. 1 to 4, a method of screening breast cancer in accordance with the invention comprises the steps of collecting a serum sample from breast cancer patients; measuring WISP1 level in the serum sample; and comparing the serum WISP1 level of healthy persons with the WISP1 level in the serum sample of the breast cancer patients. For a healthy person, mean serum WISP1 level is 631.5 pg/ml. For a patient having breast cancer, mean serum WISP1 level is 934.5 pg/ml. It is highly possible that a subject may have breast cancer if WISP1 level in his or her serum is equal to or greater than 934.5 pg/ml. Thus, the WISP1 level of 934.5 pg/ml can be used a biomarker in determining whether the subject has high risk of breast cancer occurrence or reoccurrence or not.

The measurement of serum WISP1 level is done by using Enzyme-linked immunosorbent assay (ELISA) kit (Human WISP-1/CCN4 DuoSet ELISA kit, R&D Systems) for measurement human serum WISP1 level and involves the following steps of:

pouring blood collected from a subject into a reagent bottle containing ethylenediaminetetraacetic acid disodium salt dehydrate (EDTA-2Na).

centrifuging the reagent bottle for 3,000 g, 15 minutes (i.e., blood fractionation);

drawing 100 μl liquid from an upper phase of the reagent bottle (i.e., collecting serum);

pouring the 100 μl serum into a well (Petri dish);

putting the well on a shaker for a chemical reaction for 2.5 hours at room temperature;

removing liquid out of the well;

adding 300 μl 1× wash buffer to the well for three times of cleaning;

removing liquid out of the well;

adding 100 μl biotinylated antibody to the well;

putting the well on the shaker for a chemical reaction for 1 hour at room temperature;

removing liquid out of the well;

adding 300 μl 1× wash buffer to the well for three times of cleaning;

removing liquid out of the well;

adding 100 μl streptavidin solution to the well;

putting the well on the shaker for a chemical reaction for 45 minutes at room temperature;

removing liquid out of the well;

adding 300 μl 1× wash buffer to the well for three times of cleaning;

removing liquid out of the well;

adding 100 μl TMB one-step substrate reagent to the well;

putting the well on the shaker for a chemical reaction for 30 minutes at room temperature;

adding 50 μl stop solution to the well;

disposing the well on an enzyme-linked immunosorbent assay (ELISA) reader;

reading the well at wavelength of 450 nm; and

obtaining serum WISP1 level.

The invention has the following applications:

Breast cancer screening: in a health examination, it is highly possible that a subject may have breast cancer if his or her serum WISP1 level is equal to or greater than 934.5 pg/ml. Thus, the subject needs to take further examinations for confirming whether he or she has breast cancer or not.

Tracking after breast cancer treatment: it is highly possible that a healthy patient (i.e., one being successfully treated by removing cancerous tumors) may have breast cancer recurrence if his or her serum WISP1 level is equal to or greater than 934.5 pg/ml.

Evaluation of breast cancer treatment: after a breast cancer patient taking treatment such as surgery, chemotherapy, or targeted therapy, the patient may have his or her tumors completely removed if serum WISP1 level is less than 934.5 pg/ml. To the contrary, the patient may not have his or her tumors removed if serum WISP1 level is equal to or greater than 934.5 pg/ml. Thus, another treatment should be considered.

As shown in FIG. 1 specifically, serum WISP1 level median of 14 cancerous subjects, serum WISP1 level median of 31 healthy subjects, and P value are shown. The WISP1 level median of 31 healthy subjects is 587.8 pg/ml, and the WISP1 level median of 14 cancerous subjects is 939.5 pg/ml. It is clear that the WISP1 level average of healthy subjects much less than that of cancerous subjects.

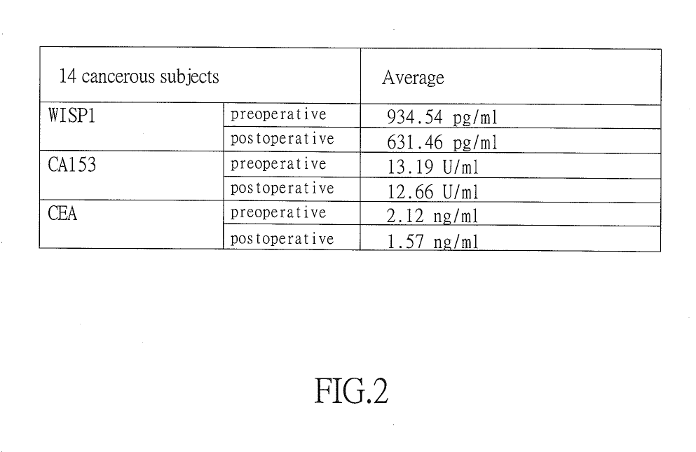

As shown in FIG. 2 specifically, preoperative and postoperative mean level of WISP1, cancer antigen 15-3 (CA153), and carcino-embryonic antigen (CEA) in the serum of 14 cancerous subjects are shown. Mean level of CA153 of the cancerous subjects is 13.19 U/ml before surgery and that of CA153 of the cancerous subjects is 12.66 U/ml after surgery. Normally, mean level of CA153 in blood is less than 38 U/ml. Thus, taking mean level of CA153 as a biomarker of breast cancer is not desirous. Mean level of CEA of the cancerous subjects is 2.12 ng/ml before surgery and that of CEA of the cancerous subjects is 1.57 ng/ml after surgery. Normally, mean level of CEA in blood is less than 10 ng/ml. Thus, taking mean level of CEA as a biomarker of breast cancer also is not desirous. Mean level of WISP1 of the cancerous subjects is 934.54 pg/ml before surgery and that of WISP1 of the cancerous subjects is 631.46 pg/ml after surgery.

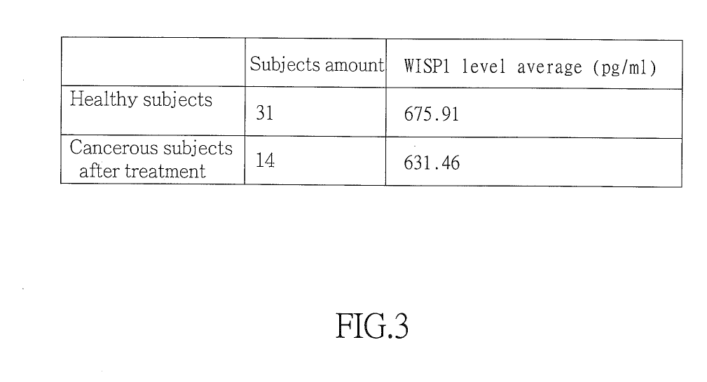

As shown in FIGS. 2 and 3 specifically, the mean serum WISP1 level of healthy persons is 675.91 pg/ml, and after surgery the mean serum WISP1 level of cancerous patients is 631.46 pg/ml down from 934.54 pg/ml. There is a significant reduction of the mean serum WISP1 level of cancerous patients before and after surgery, and after surgery the mean serum WISP1 level average of cancerous patients is near the mean serum WISP1 level average (e.g., 675.91 pg/ml) of healthy persons. It is clear that the serum WISP1 level can be taken as a good biomarker of breast cancer.

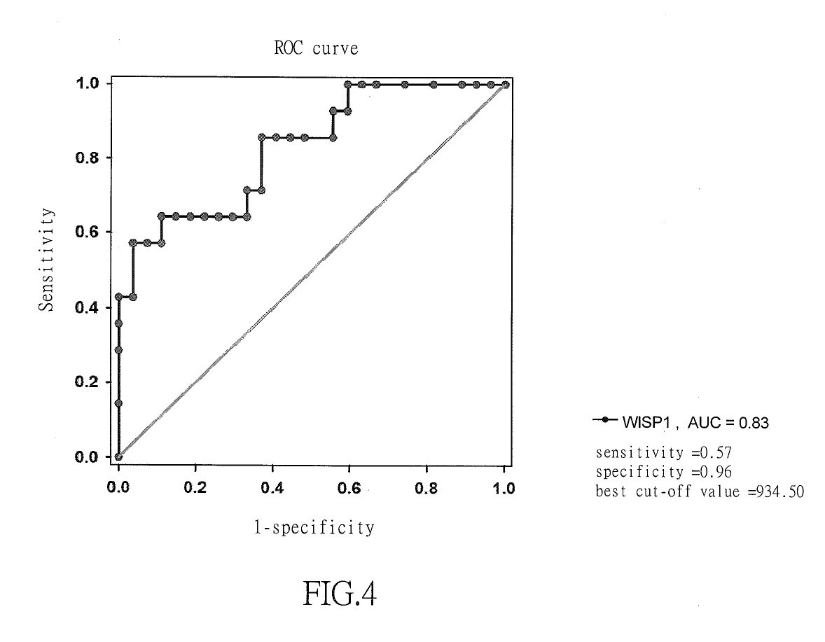

As shown in FIG. 4 ROC curves for serum WISP1 levels is constructed by comparing the healthy persons with the breast cancer patients according to the invention. Area under the curve (AUC) is 0.83 in which the curve is a receiver operating characteristic (ROC) curve. This means that serum WISP1 level can be taken as a good biomarker of breast cancer. Best cut-off value of serum WISP1 is 934.50 pg/ml, sensitivity for diagnosing breast cancer is 57%, and specificity for diagnosing breast cancer is 96%. It is clear from the tests that serum WISP1 level average of cancerous patients is greater than that of healthy subjects. After treatment, serum WISP1 level average of cancerous subjects is near that of healthy subjects. It is concluded that WISP1 level can be taken as a good biomarker of breast cancer so that the breast cancer screening for determining whether a person having high risk of breast cancer occurrence or having high possibility of cancer recurrence after a successful treatment or not can be made correct.

While the invention has been described in terms of preferred embodiments, those skilled in the art will recognize that the invention can be practiced with modifications within the spirit and scope of the appended claims.

What is claimed is:

1. A method of screening breast cancer comprising the steps of:
collecting serum samples from a breast cancer patients;
measuring the WISP1 level in the serum sample; and
comparing the serum WISP1 level of healthy persons with the WISP1 level in the serum sample of the breast cancer patients;
wherein mean serum WISP1 level average of the healthy persons is 631.5 pg/ml and the mean serum WISP1 level is 934.5 pg/ml for the breast cancer patients, so that the serum WISP1 level of 934.5 pg/ml is configured to be a biomarker in determining whether a subject has risk of breast cancer occurrence or reoccurrence or not by comparing the serum WISP1 level of the subject with the serum WISP1 level of 934.5 pg/ml.

2. The method of screening breast cancer of claim 1, wherein the serum WISP1 level of a subject having breast cancer less than 934.5 pg/ml means that a treatment of surgery, chemotherapy, or targeted therapy for the subject having breast cancer is effective.

3. The method of screening breast cancer of claim 1, wherein the serum WISP1 level of the subject having breast cancer equal or greater than 934.5 pg/ml means that a treatment of surgery, chemotherapy, or targeted therapy for the subject having breast cancer is ineffective, and the subject has risk of breast cancer recurrence and metastasis.

4. The method of screening breast cancer of claim 1, wherein the method is used to evaluate the breast cancer treatment response in the breast cancer patients and recovery therefrom.

5. The method of screening breast cancer of claim 1, wherein the measurement of the WISP1 level in the serum sample is done by using Enzyme-linked immunosorbent assay (ELISA).
